(19) United States
(12) Patent Application Publication
(10) Pub. No.: US 2012/0029375 A1
(43) Pub. Date: Feb. 2, 2012

(54) Respirations Activity and Motion Measurement Using Accelerometers

(75) Inventors: John A. Lane, Weedsport, NY (US); Henry Joseph Smith, III, Auburn, NY (US); David E. Quinn, Auburn, NY (US); Tyson B. Whitaker, Arden, NC (US)

(73) Assignee: WELCH ALLYN, INC., Skaneateles Falls, NY (US)

(21) Appl. No.: 12/848,417

(22) Filed: Aug. 2, 2010

(51) Int. Cl. A61B 5/113; G01P 15/00

(52) U.S. Cl. 600/534; 702/141

(57) ABSTRACT

A system for estimating respirations includes an accelerometer configured to be coupled to an individual's torso, the accelerometer being configured to measure a tilt of the accelerometer as the individual breathes, and a processor connected to the accelerometer, the processor being programmed to process tilt data from the accelerometer and to estimate a respiration rate based on the tilt data.

### BACKGROUND

There is currently no low cost, accurate system to measure respirations (i.e., breaths per minute). In high acuity situations, a carbon dioxide monitor, including a nasal and/or mouth cannula, can be used to estimate respirations by monitoring airflow into and out of an individual's mouth or nose. However, such machines are complex, costly, and invasive. Otherwise, in lower acuity situations, a crude manual estimate of respirations can be made by a caregiver observing the speed and level of respirations of the individual. However, such a manual estimate is prone to inaccuracy and is incomplete, since the measurement is subjective and conducted only for the duration of time that the individual is observed by the caregiver.

### SUMMARY

Embodiments of the present disclosure are directed to systems and methods for estimating respirations for an individual. In some examples described herein, an accelerometer is coupled to the individual to estimate respirations of the individual.

In one aspect, a system for estimating respirations includes: an accelerometer configured to be coupled to an individual's torso, the accelerometer being configured to measure a tilt of the accelerometer as the individual breathes; and a processor connected to the accelerometer, the processor being programmed to process tilt data from the accelerometer and to estimate a respiration rate based on the tilt data.

In another aspect, a method for estimating respirations includes: coupling a tilt sensor to a torso of an individual; measuring tilt data from the tilt sensor as the individual breathes; and estimating a respiration rate of the individual based on the tilt data.

In yet another aspect, a computer-readable data storage media encodes instructions that, when executed by a processor, cause the processor to: receive tilt data from a tilt sensor coupled to a torso of an individual; monitor the tilt data as the individual breathes; and estimate a respiration rate of the individual based on the tilt data.

### DESCRIPTION OF THE DRAWINGS

The present disclosure can be better understood with reference to the description below. Within the drawings, like reference numbers are used to indicate like parts throughout the various views. Differences between like parts may cause those like parts to be each indicated by different reference numbers. Unlike parts are indicated by different reference numbers.

FIG. 1 shows an example system for estimating respirations.

FIG. 2 shows an example sensor from the system of FIG. 1.

FIG. 3 shows the sensor of FIG. 2.

FIG. 4 shows the sensor and an example monitor from the system of FIG. 1.

FIG. 5 shows an example method for estimating respirations.

FIG. 6 shows another example method for estimating respirations.

### DETAILED DESCRIPTION

Embodiments of the present disclosure are directed to systems and methods for estimating respirations for an individual. In some examples described herein, an accelerometer is coupled to the individual to estimate respirations of the individual.

Accelerometers can be used to measure both dynamic and static measurements of acceleration. Tilt is a static measurement. When measuring tilt, gravity is the acceleration that is measured. To achieve a tilt measurement, a low-gravity, high-sensitivity accelerometer is desirable. Such accelerometers experience acceleration in the range of +1 g to −1 g as the device is tilted from −90 degrees to +90 degrees.

By using an accelerometer to measure tilt, an individual's respirations can be estimated. For example, in some embodiments, a wearable accelerometer is used that measures multi-axis movement and multi-axis acceleration. The accelerometer is positioned on the individual's torso, and the accelerometer is used to measure the tilt of the accelerometer as the individual's chest moves during respirations. This tilt information is, in turn, used to estimate the respirations of the individual.

In one embodiment, the accelerometer is a DC-response sensor that measures tilt. As the orientation of the accelerometer changes (i.e., is tilted), the impact of gravity on the accelerometer changes in a cyclical and repeatable manner, and the accelerometer can be configured to measure this change. By processing the tilt patterns measured by the accelerometer, the individual's respirations are estimated.

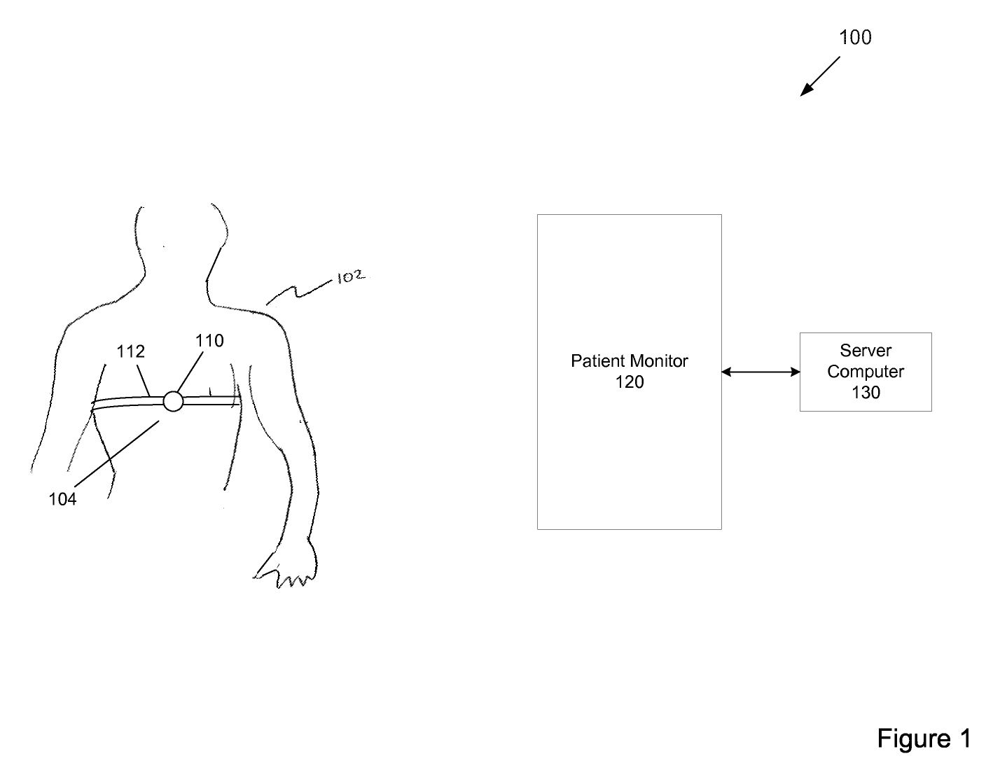

FIGS. 1-3 show an example system 100 including a sensor 110, a monitor 120, and a server computer 130.

In the example shown, the sensor 110 is an accelerometer that is coupled to a strap 112 that is positioned around the torso 104 of an individual 102. In this example, the sensor 110 is a multi-axis accelerometer, such as an ADXL325 accelerometer manufactured by Analog Devices of Norwood, Me.

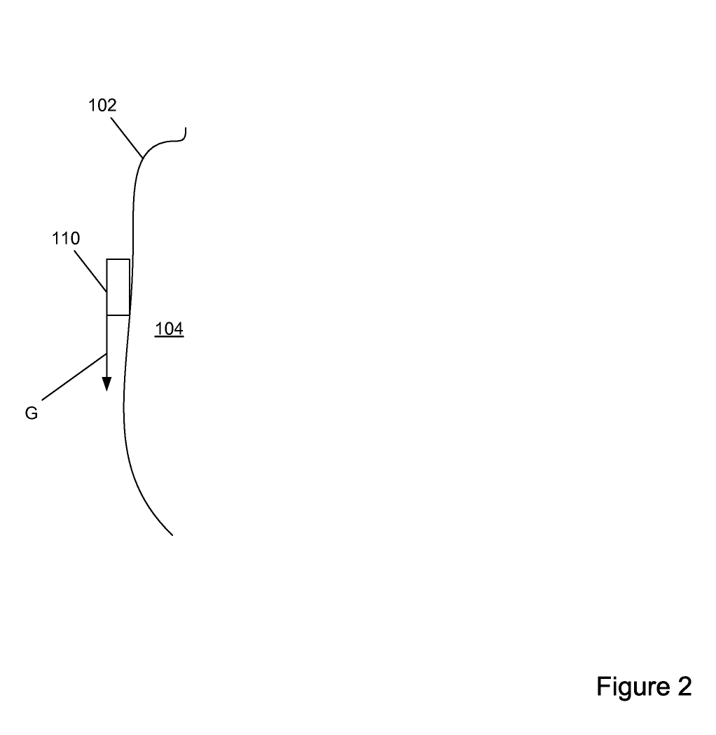

The sensor 110 is programmed to measure the tilt of the sensor 110 as the individual 102 breaths. For example, as shown in FIG. 2, the sensor 110 is positioned against the torso 104 of the individual, such as at the chest. In such an orientation, the force of gravity acting in a direction G can be measured by the sensor 110 throughout a respiration cycle.

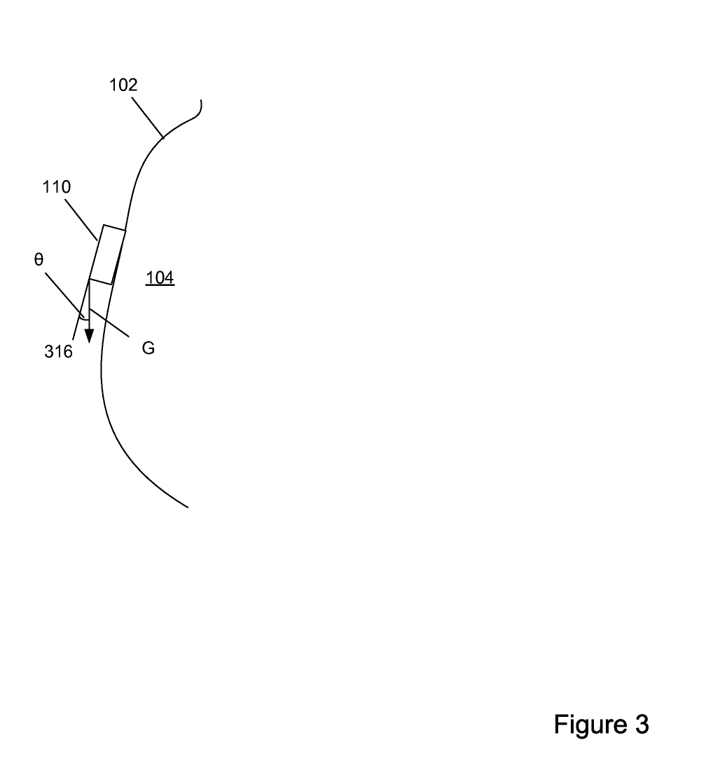

Referring now to FIG. 3, as the individual breaths, the individual's lungs fill with air, causing the individual's torso to expand. Upon expansion, the orientation of the sensor 110 is changed. Upon such a tilt, a sensing axis 316 of the sensor 110 is changed, and an angle θ is formed between the sensing axis 316 and the force of gravity in the direction G.

By monitoring this periodic tilt of the sensor 110 as the individual breaths, the sensor 110 can be used to estimate a rate of respiration. Further, by estimating the magnitude of the angle θ, the quality of each of the breaths (e.g., shallow, deep, etc.) can also be estimated. The sensor 110 can also be programmed to measure the dynamic acceleration of the sensor 110 as the individual breathes to supplement the estimate of the rate and/or quality of respirations.

Further, the sensor 110 can be used to estimate a status of the individual as the respiration measurements are taken. For example, an estimate of whether the individual is prone or upright can be made as the respiration estimates are taken. In addition, an estimate of whether the individual is moving or still can also be made. If moving, an estimate of the type of movement (e.g., ascending, descending, level) can also be made. These characteristics can be examined as the respirations estimates are made to provide further detail to supplement the data. For example, the respirations may increase as the individual runs or ascends a large hill. Other configurations are possible.

Referring again to FIG. 1, in the examples shown, the sensor 110 communicates with the monitor 120 using wireless or wired technologies. For example, in one embodiment, the sensor 110 communicates with the monitor 120 using a wireless protocol such as Bluetooth. Additional details of the pairing of the sensor 110 with the monitor 120 are disclosed in U.S. patent application Ser. No. 12/723,726 filed on Mar. 15, 2010 and U.S. patent application Ser. No. 12/827,817 filed on Jun. 30, 2010, the entireties of which are hereby incorporated by reference. Other configurations are possible.

Once paired, in a first embodiment, the sensor 110 sends data related to the tilting of the sensor 110 to the monitor 120. The monitor 120, in turn, estimates respirations based on the tilt data from the sensor 110. In a second embodiment, the sensor 110 is programmed to estimate respirations based on the data sensed by the sensor 110. The sensor 110 thereupon sends the estimated respirations to the monitor 120.

The monitor 120 can display the estimated respiration data to a user, such as a caregiver. For example, the monitor 120 can display the rate of respiration (e.g., breaths per minute), the quality of respiration (e.g., shallow, deep, etc.), and/or the activity level of the individual (e.g., lying down, upright, moving, etc.).

In the examples shown, the monitor 120, in turn, communicates with a server computer 130. The monitor 120 can communicate the respiration data to the server computer 130. The server computer 130 can further process the data, display the data to a caregiver (e.g., at a central monitoring station in a hospital), and/or store the data (e.g., in the individuals electronic medical record).

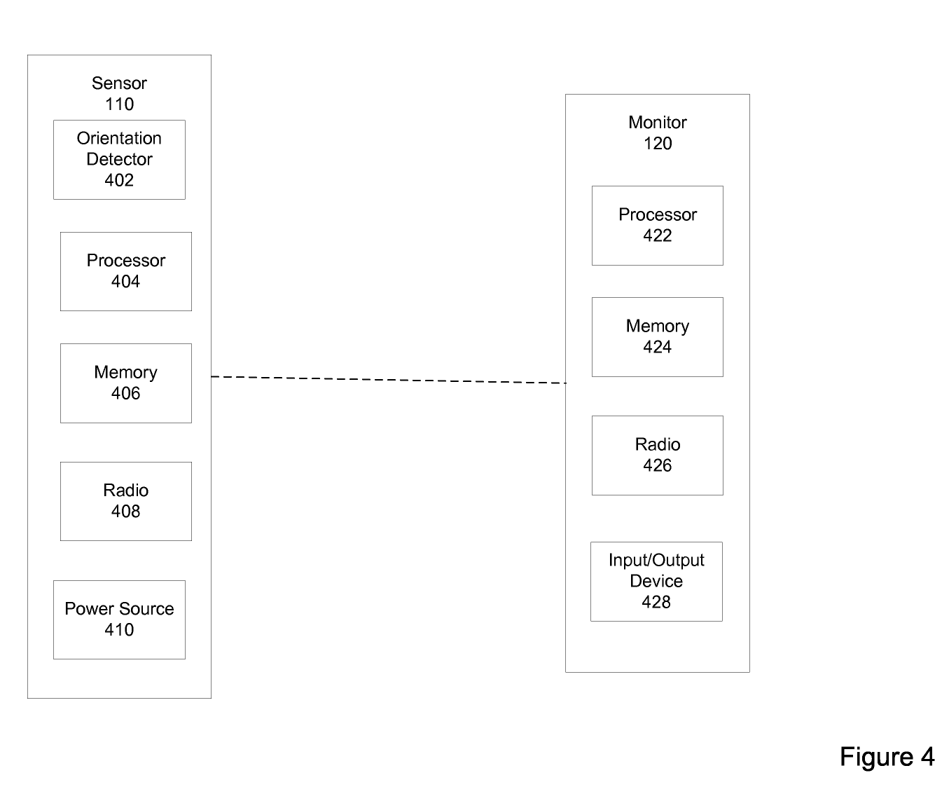

Referring now to FIG. 4, additional details about the sensor 110 and the monitor 120 are shown.

The sensor 110 including an orientation detector 402, an optional processor 404, a memory 406, a radio 408, and a power source 410.

The orientation detector 402 is the accelerometer that can measure the tilt and/or acceleration of the sensor 110. In one example, the orientation detector is a three-dimensional accelerometer. The orientation detector 402 is configured to measure orientation at given intervals, such as every 20 seconds, once per minute, once per hour, once every hour, etc. For example, the orientation detector 402 is configured to estimate a tilt and/or an orientation of the sensor 110, as described herein, to estimate respirations and/or body position. The orientation detector 402 can be set to custom sampling times (e.g., spot check versus monitoring) and to average times depending on the application.

In one example, the orientation detector 402 samples three difference axis (X, Y, Z) of orientation to determine what position the individual is in and what axis is predominate to extract respiratory cycles. The relevant axis can vary depending on the position of the individual (i.e., lying down versus sitting up versus angled in bed, etc.). A most relevant axis can be derived from a combination of X, Y, and Z.

In some examples, the sensor 110 includes the processor 404 that takes the orientation data from the orientation detector 402 and can manipulate the data to estimate respirations and body position. For example, the processor 404 can obtain the tilt data from the orientation detector 402 and use that tilt data to estimate a rate of respirations for the individual. In other examples, the processor 404 can be omitted, and the data from the orientation detector 402 can be sent to another device to estimate respirations (e.g., the monitor 120).

The memory 406 is used to store instructions that are executed by the processor 404, as well as to store data from the orientation detector 402. In the example shown, the memory 406 includes a random access memory (“RAM”) and/or a read-only memory (“ROM”). In some examples, the memory 406 can also include a mass storage device. The mass storage device stores software instructions and data.

The mass storage device and its associated computer-readable data storage media provide non-volatile, non-transitory storage. Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the monitor 120.

As mentioned above, the mass storage device and the RAM can store software instructions and data. The software instructions can include an operating system suitable for controlling the operation of the sensor 110. The mass storage device and/or the RAM also store software instructions, that when executed by the processor 404, cause the sensor 110 to provide the functionality of the sensor 110 described herein. For example, the mass storage device and/or the RAM can store software instructions that, when executed by the processor 404, cause the sensor 110 to monitor and estimate respirations as described herein.

The radio 408 is used to communicate with other devices, such as the monitor 120. In some examples, the radio 408 communicates using Bluetooth, although other networking protocols such as 802.15.4, ZigBee, UWB, a low-power 802.11 network, or a proprietary network could be used.

The power source 410 powers the sensor 110. In one example, the power source 410 is a battery. In another example, the power is harvested from the individual and/or is provided from an external source (e.g., mains power). Other configurations are possible.

The monitor 120 includes a processor 422, a memory 424, a radio 426, and an input/output device 428.

Similar to the sensor 110, the processor 422 of the monitor 120 is programmed to take the orientation data from the orientation detector 402 and manipulate the data to estimate respirations and body position. The memory 424 is likewise configured to store data and instructions for execution. The radio 426 is programmed for wireless communications (wired communications are also possible) with the sensor 110.

The input/output device 428 can be one or more of a number of different devices. For example, the device 428 can be a display that displays various parameters associated with respirations to the caregiver, such as rate of respiration (e.g., breaths per minute), the quality of respiration (e.g., shallow, deep, etc.), and/or the activity level of the individual (e.g., lying down, upright, moving, etc.). The device 428 can also be an input device, such as a keyboard or touch screen. Other types of possible devices include a mouse, a printer, or other type of input or output devices.

In one example, the monitor 120 is a patient monitor device such as that disclosed in U.S. patent application Ser. No. 12/751,579 filed on Mar. 31, 2010, the entirety of which is hereby incorporated by reference.

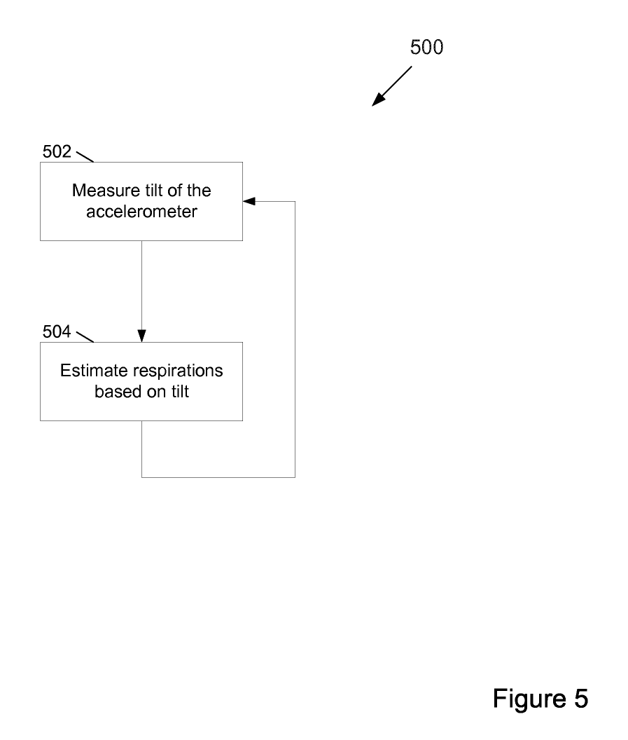

Referring now to FIG. 5, an example method 500 for estimating respirations information is shown.

Initially, at operation 502, the tilt of the sensor is measured at periodic intervals. Next, at operation 504, the tilt data is used to estimate respiration information, as described herein.

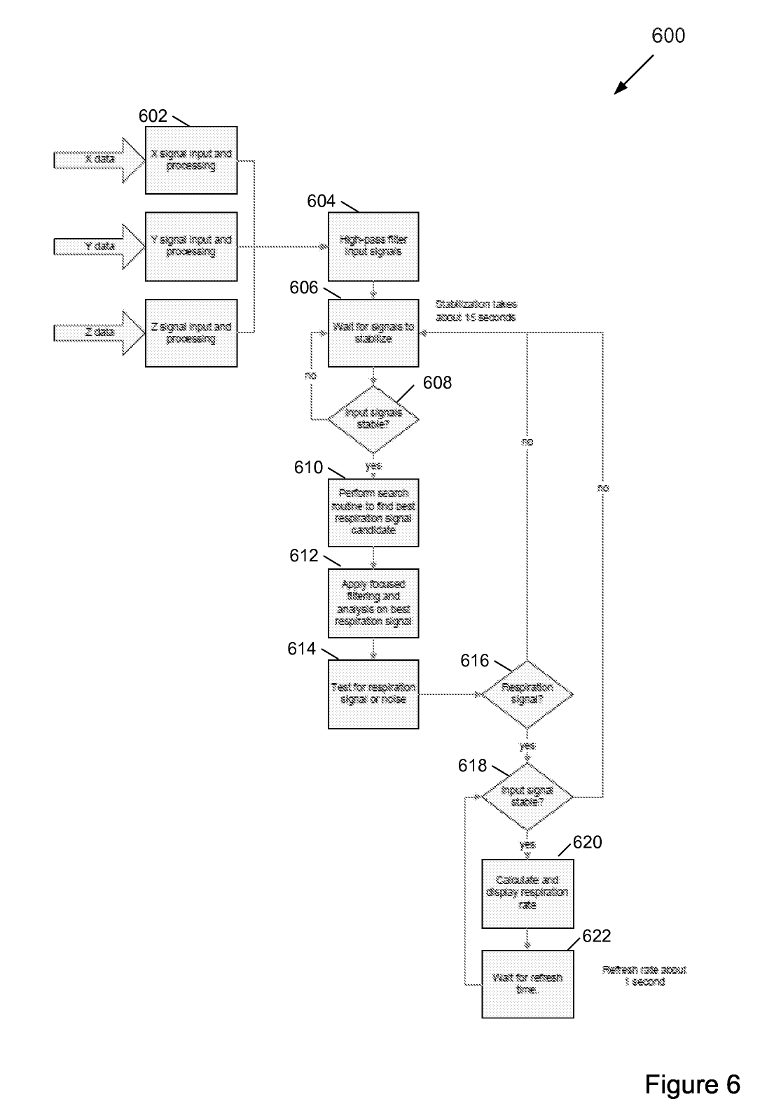

Referring to FIG. 6, another example method 600 for estimating respirations information is shown.

Initially, at operation 602, the input signals from the different axis of a multi-axis accelerometer are processed. Next, at operation 604, the signals are run through a high-pass filter to reduce extraneous noise.

Next, at operation 606, a pause is used to wait for the signals to stabilize. For example, in some embodiments, the pause is about 1 second, 5 seconds, 10 seconds, 15 seconds, 20 seconds, or 30 seconds. Next, at operation 608, a determination is made as to whether or not the signals have stabilized. If the signals have not stabilized, control is passed back to operation 606 for additional waiting.

Otherwise, control is passed to operation 610, at which a search routine is used to determine the best respiration signal candidate from the various input signals (i.e., the signals from the X, Y, and Z axis). In one example, a band-pass filter is applied within the ¼ to ½ hertz range in an attempt to find the best respiration signal candidate. Signals falling below that range are typically associated with gravity, and signals above the range are typically associated with activity.

Next, at operation 614, a test is performed to determine if the signal is likely respiration or noise. One example of such as test is to determine if there are multiple occurrences of cyclical changes having a period that represents a reasonable human respiration rate. At operation 616, a determination is made regarding whether the signal is a respiration candidate. If not, control is passed back to operation 606.

If so, control is instead passed to operation 620, where the rate of respiration is estimated, and, in some embodiments, the rate is displayed for the caregiver to review. Next, at operation 622, a refresh pause is instituted, such as 0.5 seconds, 1 second, 2 seconds, or 5 seconds depending on how quickly updating of the display is desired. Finally, after the refresh pause, control is passed back to operation 618.

Various embodiments disclosed herein can be implemented (1) as a sequence of computer implemented acts or program modules running on a computing system and/or (2) as interconnected machine logic circuits or circuit modules within the computing system. The implementation is a matter of choice dependent on the performance requirements of the computing system. Accordingly, logical operations including related algorithms can be referred to variously as operations, structural devices, acts or modules. It will be recognized by one skilled in the art that these operations, structural devices, acts and modules may be implemented in software, firmware, special purpose digital logic, and any combination thereof without deviating from the spirit and scope of the present disclosure.

Although the disclosure has been described in connection with various embodiments, those of ordinary skill in the art will understand that many modifications may be made thereto. Accordingly, it is not intended that the scope of the disclosure in any way be limited by the above description.

What is claimed is:

1. A system for estimating respirations, the system comprising:
an accelerometer configured to be coupled to an individual's torso, the accelerometer being configured to measure a tilt of the accelerometer as the individual breathes; and
a processor connected to the accelerometer, the processor being programmed to process tilt data from the accelerometer and to estimate a respiration rate based on the tilt data.

2. The system of claim 1, wherein the accelerometer is a three-axis accelerometer.

3. The system of claim 2, wherein the processor is further programmed to estimate a body position of the individual based on the tilt data.

4. The system of claim 3, wherein the processor is further programmed to estimate a quality of breathing based on the tilt data.

5. The system of claim 1, wherein the processor is further programmed to estimate a body position of the individual based on the tilt data.

6. The system of claim 1, wherein the processor is further programmed to estimate a quality of breathing based on the tilt data.

7. The system of claim 1, wherein the accelerometer is further configured to estimate a dynamic acceleration of the accelerometer as the individual breathes.

8. A method for estimating respirations, the method comprising:
coupling a tilt sensor to a torso of an individual;
measuring tilt data from the tilt sensor as the individual breathes; and
estimating a respiration rate of the individual based on the tilt data.

9. The method of claim 8, further comprising:
filtering a plurality of signals from the tilt sensor;
searching for a candidate from the plurality of signals;
testing the candidate; and
when the candidate passes the testing, estimating the respiration rate based on tilt data associated with the candidate.

10. The method of claim 8, further comprising using a three-axis accelerometer as the tilt sensor.

11. The method of claim 10, further comprising estimating a body position of the individual based on the tilt data.

12. The method of claim 11, further comprising estimating a quality of breathing based on the tilt data.

13. The method of claim 10, further comprising estimating a dynamic acceleration of the three-axis accelerometer as the individual breathes.

14. The method of claim 8, further comprising estimating a body position of the individual based on the tilt data.

15. The method of claim 8, further comprising estimating a quality of breathing based on the tilt data.

16. A computer-readable data storage media encoding instructions that, when executed by a processor, cause the processor to:
receive tilt data from a tilt sensor coupled to a torso of an individual;
monitor the tilt data as the individual breathes; and
estimate a respiration rate of the individual based on the tilt data.

17. The computer-readable data storage media of claim 16, wherein the computer-readable data storage media encodes further instructions that, when executed by the processor, cause the processor to:
filter a plurality of signals from the tilt sensor;
search for a candidate from the plurality of signals;
test the candidate; and
when the candidate passes the testing, estimate the respiration rate based on tilt data associated with the candidate.

18. The computer-readable data storage media of claim 17, wherein the computer-readable data storage media encodes further instructions that, when executed by the processor, cause the processor to estimate a body position of the individual based on the tilt data.

19. The computer-readable data storage media of claim 18, wherein the computer-readable data storage media encodes further instructions that, when executed by the processor, cause the processor to estimate a quality of breathing based on the tilt data.

20. The computer-readable data storage media of claim 16, wherein the computer-readable data storage media encodes further instructions that, when executed by the processor, cause the processor to estimate a body position of the individual based on the tilt data.
